(19) United States
(12) Patent Application Publication
(10) Pub. No.: US 2021/0341487 A1
(43) Pub. Date: Nov. 4, 2021

(54) NEUTRALIZING ANTIBODY ASSAY FOR THERAPEUTIC PROTEINS

(71) Applicant: Regeneron Pharmaceuticals, Inc., Tarrytown, NY (US)

(72) Inventors: Michael Partridge, Eastchester, NY (US); Susan Irvin, Thornwood, NY (US); Manoj Rajadhyaksha, Colchester, CT (US); Aynur Hermann, New York, NY (US)

(21) Appl. No.: 17/245,271

(22) Filed: Apr. 30, 2021

Related U.S. Application Data
(60) Provisional application No. 63/018,821, filed on May 1, 2020.
(60) Provisional application No. 63/041,768, filed on Jun. 19, 2020.
(60) Provisional application No. 63/172,488, filed on Apr. 8, 2021.

(51) Int. Cl. G01N 33/574; G01N 33/50

(52) U.S. Cl. CPC G01N 33/57492; G01N 33/5023; G01N 33/5041

(57) ABSTRACT

The present invention generally pertains to methods of testing for the presence of neutralizing antibodies (NAbs) against therapeutic proteins. In particular, the present invention pertains to the use of mitigating agents against interfering competing drugs in ligand binding assays or cell-based assays for the detection of neutralizing antibodies against therapeutic proteins.

### CROSS-REFERENCE TO RELATED APPLICATIONS

This application claims priority to and the benefit of U.S. Provisional Patent Application No. 63/018,821, filed May 1, 2020, U.S. Provisional Patent Application No. 63/041,768, filed Jun. 19, 2020 and U.S. Provisional Patent Application No. 63/172,488 filed Apr. 8, 2021 which are each herein incorporated by reference.

### FIELD

This application relates to assay methods, modules, and kits for conducting diagnostic assays for detection of neutralizing antibodies against therapeutic proteins.

### BACKGROUND

Administration of biological therapeutics to a patient can induce an undesirable immunogenic response in the patient that can lead to the development of anti-drug antibodies (ADAs) (Mire-Sluis, A. R., et al., J Immunol Methods, 289(1):1-16 (2004)). Neutralizing antibodies (NAbs) are a subset of ADAs that inhibit binding of the drug to its target, rendering the drug biologically inactive. By definition, NAbs neutralize the effect of the drug, potentially reducing clinical activity. In addition, where the drug is a biological mimic of an endogenous protein, NAbs may cross-react with the drug's endogenous analogue, which can have critical consequences for drug safety (Finco, D., et al., J Pharm Biomed Anal, 54(2):351-358 (2011); Hu, J., et al., J Immunol Methods, 419:1-8 (2015)).

Detection of an immunogenic response involves a tiered approach where a sample is first tested for the presence of ADAs, typically using a bridging immunoassay (Mire-Sluis, A. R., et al., J Immunol Methods, 289(1):1-16 (2004)). Further characterization of the ADA response may include a titer assay to determine the relative amount of ADAs, and an assay to determine whether the antibody response is neutralizing (Wu, B., et al., AAPS Journal, 18(6):1335-1350 (2016); Shankar, G, et al., J Pharm Biomed Anal 48(5):1267-1281 (2008); Gupta, S., et al., J Pharm Biomed Anal, 55(5):878-888 (2011)).

NAb assays can be subject to interference that prevents accurate quantitation of neutralization against the therapeutic protein. For example, if the endogenous drug target is soluble, it may be present in the subject sample and competitively bind with the therapeutic, creating a false positive NAb signal. There may also be residual drug in the subject sample from previous administrations of the therapeutic, which can competitively bind to NAbs and create a false negative NAb signal. Different techniques have been developed to deal with these sources of interference to obtain an accurate quantitation of NAbs (Xu, W., et al., J Immunol Methods, 462:34-41 (2018); Xu, W., et al., J Immunol Methods, 416:94-104 (2015); Xiang, Y., et al., AAPS Journal, 21(1):4 (2019); Sloan, J. H., et al., Bioanalysis, 8(20):2157-2168 (2016)).

An additional source of potential interference that has not yet been characterized is interference by a residual drug, different from the therapeutic protein being tested, that competitively binds to the same drug target as the therapeutic, which would create a false positive NAb signal. As such, a strategy to mitigate this type of interference has also not been developed to date.

Therefore, it will be appreciated that a need exists for methods to identify and mitigate interference from competing drugs in ligand binding assays or cell-based assays for the detection of neutralizing antibodies against therapeutic proteins.

### SUMMARY

This disclosure provides a method for detecting a neutralizing agent to a therapeutic protein in a sample. In some exemplary embodiments, the method comprises (a) contacting said sample having said neutralizing agent and a competing drug to (i) said therapeutic protein, (ii) a target of said therapeutic protein, and (iii) a mitigating agent; (b) measuring a binding of said therapeutic protein to said target; and (c) comparing the result of (b) to a control measurement to detect said neutralizing agent.

In one aspect, said control measurement is obtained by measuring binding of said therapeutic protein to said target in the absence of a neutralizing agent. In another aspect, said neutralizing agent is a neutralizing antibody.

In one aspect, said therapeutic protein is an antibody, a soluble receptor, an antibody-drug conjugate, or an enzyme. In a specific aspect, said therapeutic protein is a monoclonal antibody. In yet another specific aspect, said monoclonal antibody is an anti-PD-1 antibody, an anti-TNF antibody, an anti-PD-L1 antibody, an anti-EGFR antibody, an anti-CD20 antibody, an anti-CD38 antibody, or an anti-LAG3 antibody.

In one aspect, said therapeutic protein is a bispecific antibody. In a specific aspect, said bispecific antibody is a CD20×CD3 antibody, a BCMA×CD3 antibody, a EGFR×CD28 antibody, or a CD38×CD28 antibody.

In one aspect, said therapeutic protein is immobilized to a solid support. In another aspect, said therapeutic protein is labeled for detection. In a specific aspect, said label is detectable by fluorescence, chemiluminescence, electrochemiluminescence, radioactivity, or affinity purification. In yet another specific aspect, said label comprises ruthenium.

In one aspect, said target is an antigen, a receptor, a ligand, or an enzymatic substrate. In another aspect, said target is a cell surface protein. In yet another aspect, said target is a recombinant protein. In yet another aspect, said target is expressed by a cell. In a specific aspect, said cell is a HEK293 cell, a MOLP-8 cell, a Jurkat cell, or a modified version thereof.

In one aspect, said target is immobilized to a solid support. In another aspect, said target is labeled for detection. In a specific aspect, said label is detectable by fluorescence, chemiluminescence, electrochemiluminescence, radioactivity, or affinity purification. In another aspect, said target is an enzymatic substrate. In yet another aspect, said target is CD20, CD3, BCMA, PD-1, EGFR, CD28, CD38, TNF, PD-L1, or LAG3. In yet another aspect, said method additionally comprises a second target.

In one aspect, said competing drug is a monoclonal antibody. In a specific aspect, said competing drug is rituximab, pembrolizumab, nivolumab, ocrelizumab, obinutuzumab, ofatumumab, ibritumomab tiuxetan, tositumomab, ublituximab, cetuximab, daratumumab, or adalimumab. In another aspect, said competing drug is a bispecific antibody.

In one aspect, said mitigating agent is a monoclonal antibody. In another aspect, said method comprises using two, three, four or more mitigating agents.

In one aspect, a binding of said therapeutic protein to said target is measured by measuring receptor phosphorylation, phosphorylation of downstream proteins in a signal transduction pathway, cytokine release, cell proliferation, cell death, or production of a secondary protein. In another aspect, a binding of said therapeutic protein to said target is measured by the expression of a reporter gene. In a specific aspect, said reporter gene is luciferase.

In one aspect, said method further comprises a pre-treatment step of contacting said sample to said mitigating agent prior to contacting said sample to said therapeutic protein or said target.

This disclosure also provides a kit for carrying out the method of the invention. In some exemplary embodiments, the kit comprises a therapeutic protein, a target of said therapeutic protein, a neutralizing agent against said therapeutic protein, a competing drug, and a mitigating agent.

In one aspect, said kit further comprises cells that express said target. In a specific aspect, said kit further comprises cells that produce a measurable activity or signal in response to the binding of said therapeutic protein to said target. In another specific aspect, said activity is the expression of luciferase.

In one aspect, said target is immobilized to a solid support. In another aspect, said kit further comprises a label affixed to said therapeutic protein. In a specific aspect, said label comprises ruthenium.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A shows a diagram of a cell-based neutralizing antibody (NAb) assay according to an exemplary embodiment. FIG. 1B shows an increase in luciferase activity with increasing concentrations of a bispecific CD20×CD3 drug antibody, while a negative control antibody induces no luciferase signal according to an exemplary embodiment. FIG. 1C shows an increase in luciferase activity with increasing concentrations of two bispecific BCMA×CD3 drug antibodies according to an exemplary embodiment.

FIG. 2A shows a diagram of a cell-based NAb assay with the addition of neutralizing antibodies against each arm of a therapeutic antibody according to an exemplary embodiment. FIG. 2B shows a decrease in luciferase activity with increasing concentrations of surrogate neutralizing antibodies against either the CD20 arm or the CD3 arm of a bispecific CD20×CD3 drug antibody according to an exemplary embodiment.

FIG. 2C shows a decrease in luciferase activity with increasing concentrations of surrogate neutralizing antibodies against the BCMA arm of a bispecific BCMA×CD3 drug antibody according to an exemplary embodiment. FIG. 2D shows a decrease in luciferase activity with increasing concentrations of surrogate neutralizing antibodies against the CD3 arm of a bispecific BCMA×CD3 drug antibody according to an exemplary embodiment. FIG. 2E shows no change in luciferase activity with the addition of isotype control antibodies to a NAb assay for a bispecific BCMA×CD3 drug antibody according to an exemplary embodiment.

FIG. 2F shows a decrease in luciferase activity with increasing concentrations of surrogate neutralizing antibodies against the BCMA arm of a second bispecific BCMA×CD3 drug antibody according to an exemplary embodiment. FIG. 2G shows a decrease in luciferase activity with increasing concentrations of surrogate neutralizing antibodies against the CD3 arm of a second bispecific BCMA×CD3 drug antibody according to an exemplary embodiment. FIG. 2H shows no change in luciferase activity with the addition of isotype control antibodies to a NAb assay for a second bispecific BCMA×CD3 drug antibody according to an exemplary embodiment.

FIG. 3A shows a decrease in luciferase activity in a NAb assay for a bispecific CD20×CD3 drug antibody with the addition of competing antibodies against the drug target CD20 according to an exemplary embodiment. FIG. 3B shows a decrease in luciferase activity in a NAb assay for a bispecific CD20×CD3 drug antibody with the addition of competing antibodies against the drug target CD3 according to an exemplary embodiment. FIG. 3C and FIG. 3D show a decrease in luciferase activity in a NAb assay for a bispecific BCMA×CD3 drug antibody with the addition of competing antibodies against the drug targets BCMA or CD3 according to an exemplary embodiment. FIG. 3E and FIG. 3F show a decrease in luciferase activity in a NAb assay for a second bispecific BCMA×CD3 drug antibody with the addition of competing antibodies against the drug targets BCMA or CD3 according to an exemplary embodiment.

FIG. 4A shows an increase in luciferase activity in a NAb assay with increasing concentrations of therapeutic antibody according to an exemplary embodiment. The addition of naïve human serum had no effect on luciferase activity. FIG. 4B illustrates the quantification of NAb assay signal by comparing luciferase activity in the presence of drug control to luciferase activity in the presence of experimental sample according to an exemplary embodiment.

FIG. 5 shows cell-based NAb assay results from 60 drug-naïve clinical samples according to an exemplary embodiment.

FIG. 6 shows a correlation between concentration of rituximab in clinical samples and NAb assay signal according to an exemplary embodiment.

FIG. 7A shows a diagram of a cell-based NAb assay with the addition of rituximab according to an exemplary embodiment. FIG. 7B shows a diagram of the NAb assay with the addition of rituximab and mitigating antibodies against rituximab according to an exemplary embodiment. FIG. 7C shows the restoration of luciferase activity in the NAb assay with the addition of mitigating antibodies against rituximab according to an exemplary embodiment.

FIG. 8 shows the reduction of false positive NAb assay signal in drug-naïve clinical samples with the addition of mitigating antibodies against rituximab according to an exemplary embodiment.

FIG. 9A shows a diagram of a target-capture ligand binding NAb assay according to an exemplary embodiment. FIG. 9B shows a diagram of the target-capture ligand binding NAb assay with the addition of NAbs against an arm of the therapeutic protein according to an exemplary embodiment. FIG. 9C shows a diagram of a drug-capture ligand binding NAb assay according to an exemplary embodiment.

FIG. 10A shows a diagram of a ligand binding NAb assay with the addition of a competing drug according to an exemplary embodiment. FIG. 10B shows an increase in false positive signal inhibition in the ligand binding NAb assay with increasing concentrations of competing drugs according to an exemplary embodiment.

FIG. 11A shows a diagram of a ligand binding NAb assay with the addition of a competing drug and mitigating antibodies against the competing drug according to an exemplary embodiment. FIG. 11B shows the elimination of false positive NAb assay signal with the addition of mitigating antibodies against competing drugs according to an exemplary embodiment.

### DETAILED DESCRIPTION

Therapeutic proteins are an important class of drugs used to treat a variety of human diseases. However, therapeutic proteins can elicit immune responses in dosed recipients, generating anti-drug antibodies (ADAs). Neutralizing antibodies (NAbs) are a subpopulation of ADAs that can potentially impact patient safety and mediate loss of drug efficacy by blocking the biological activity of a therapeutic protein. Therefore, characterizing and monitoring NAbs is an important aspect of immunogenicity assessment, requiring sensitive and reliable methods reflective of the therapeutic mechanism of action (Wu, B., et al., AAPS Journal, 18(6):1335-1350 (2016)).

NAb assays are expected to reliably detect NAbs with adequate sensitivity, specificity, selectivity, and precision. Both cell-based and non cell-based assays are options for NAb assessment. In general, a NAb assay presents a target for a therapeutic protein, and a mechanism for signal output as a response to the therapeutic protein binding to its target, allowing for quantitation of binding. If NAbs are present in a co-incubated sample, they will inhibit the binding of the therapeutic protein to the target, reducing the signal output and allowing for quantitation of NAbs in the sample.

The sample matrix may include interfering agents that prevent accurate quantitation of NAbs, for example by directly interacting with NAbs, the therapeutic protein or the target. A matrix component that may interfere by interacting with and occupying NAbs includes, for example, residual drug from a previous administration of the therapeutic protein. Another component that may interfere by interacting with and occupying the therapeutic protein includes, for example, a soluble drug target. These interfering agents have been characterized in the prior art, and techniques have been developed to deal with these sources of interference to obtain an accurate quantitation of NAbs (Xu, W., et al., J Immunol Methods, 462:34-41 (2018); Xu, W., et al., J Immunol Methods, 416:94-104 (2015); Xiang, Y., et al., AAPS Journal, 21(1):4 (2019); Sloan, J. H., et al., Bioanalysis, 8(20):2157-2168 (2016)).

However, another possible interfering agent that has not yet been characterized or addressed is a residual competing drug in a subject sample, distinct from the therapeutic protein being tested, which may interact with and occupy the target of the therapeutic protein, resulting in a false positive quantitation of NAbs.

To meet the challenges of accurately measuring neutralizing antibodies against a therapeutic protein, described herein are methods and kits for using mitigating agents against a competing drug to prevent interference in a neutralizing antibody assay. Also disclosed herein is the detection of interference in NAb assays from drugs that competitively bind to the target of a therapeutic protein. This interference can result in the reduction of therapeutic protein binding signal or activity in the NAb assay and a false positive NAb assay signal. In order to overcome this interference, mitigating agents can be employed which reduce the binding of the competing drug to the target, allowing the therapeutic protein to bind to its target, and restoring an accurate NAb assay signal.

Interference from residual competing drugs is a serious challenge in accurately assessing NAbs while testing a therapeutic protein for clinical use, as demonstrated for example in Examples 5 and 6. Novel therapeutics may be tested after patients have already been administered a first line of therapy, which may competitively interact with the same target. In these cases, interference from competing drugs must be identified and mitigated. For example, numerous drug candidates with shared targets of B-cell maturation antigen (BCMA) or CD3 are listed in Table 1. Other therapeutic targets for which there may be many competing drugs include, for example: epidermal growth factor receptor (EGFR), which may be targeted by drugs or drug candidates such as cetuximab; CD28; CD38, which may be targeted by drugs or drug candidates such as daratumumab; lymphocyte-activation gene 3 (LAG3); programmed cell death protein 1 (PD-1), which may be targeted by drugs or drug candidates such as cemiplimab, pembrolizumab, or nivolumab; programmed death-ligand 1 (PD-L1); tumor necrosis factor (TNF), which may be targeted by drugs or drug candidates such as adalimumab; or CD20, which may be targeted by drugs or drug candidates such as rituximab, ocrelizumab, obinutuzumab, ofatumumab, ibritumomab tiuxetan, tositumomab, or ublituximab. The disclosure herein teaches a method that would be suitable to mitigate NAb assay interference from these, and other, drugs and drug candidates.

TABLE 1
Examples of drug candidates with shared targets

| Drug Candidate Name | Target | Company |
|---|---|---|
| belantamab mafodotin | BCMA | Glaxo Group, Seattle |
| | | Genetics |
| JNJ-68284528 | BCMA | Janssen Biotech |
| JNJ-64007957 | BCMA, CD3 | Genmab, Janssen Biotech |
| LCAR-B38M | BCMA | Nanjing Legend Bio |
| SEA-BCMA | BCMA | Seattle Genetics |
| AMG 420 | BCMA, CD3 | Amgen, Boehringer, |
| | | Micromet |
| AMG 224 | BCMA | Amgen |
| bb2121 | BCMA | Bluebird, Celgene |
| U.Penn. anti-BCMA | BCMA | U.Penn. |
| CAR | | |
| MED12228 | BCMA | Medimmune |
| TNB-383B | BCMA, CD3 | Abbvie, TeneoBio |
| CC-93269 | BCMA, CD3 | Celgene, Engmab |
| AMG 701 | BCMA, CD3 | Amgen |
| Pregene Bio anti-BCMA | BCMA | Pregene Bio |
| CAR | | |
| BsAb A | BCMA, CD3 | Regeneron |
| HPN217 | BCMA, CD3, | Abbvie, Harpoon |
| | Serum Albumin | |
| CT053 | BCMA | Carsgen |
| CC-99712 | BCMA | Celgene |
| BsAb B | BCMA, CD3 | Regeneron |

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. A protein can include antibody fragments, nanobodies, recombinant antibody chimeras, cytokines, chemokines, peptide hormones, and the like. Proteins of interest can include any of biotherapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platformsfor biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, a protein of interest can be a recombinant protein, an antibody, a bispecific antibody, a multispecific antibody, antibody fragment, monoclonal antibody, fusion protein, scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG (e.g., IgG1, IgG2, IgG3, IgG4), IgM, IgA1, IgA2, IgD, or IgE. In certain exemplary embodiments the antibody molecule is a full-length antibody (e.g., an IgG1 or IgG4 immunoglobulin) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KX-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated).

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, a protein of interest can be produced from mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPM12650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

As used herein, the term “therapeutic protein” refers to any protein that can be administered to a subject for the treatment of a disease or disorder. In some exemplary embodiments, the therapeutic protein can be directed towards the treatment of cancer. A therapeutic protein may be any protein with a pharmacological effect, for example, an antibody, a soluble receptor, an antibody-drug conjugate, or an enzyme. In some exemplary embodiments, the therapeutic protein can be a bispecific CD20×CD3 antibody. In some exemplary embodiments, the therapeutic protein can be a bispecific BCMA×CD3 antibody. In some exemplary embodiments, the therapeutic protein can be a monoclonal antibody against programmed cell death protein 1 (PD-1), such as cemiplimab. In other embodiments, the therapeutic protein can be a bispecific EGFR×CD28 antibody, a bispecific CD38×CD28 antibody, a monoclonal anti-TNF antibody, a monoclonal anti-PD-L1 antibody, a monoclonal anti-EGFR antibody, a monoclonal anti-CD20 antibody, a monoclonal anti-CD38 antibody, or a monoclonal anti-LAG3 antibody.

As used herein, the term “target” refers to any molecule that may specifically interact with a therapeutic protein in order to achieve a pharmacological effect. For example, the target of an antibody may be an antigen against which it is directed; the target of a ligand may be a receptor to which it preferentially binds, and vice versa; the target of an enzyme may be a substrate to which it preferentially binds; and so forth. A single therapeutic protein may have more than one target. A variety of targets are suitable for use in the method of the invention, according to the specific application. A target may, for example, be present on a cell surface, may be soluble, may be cytosolic, or may be immobilized on a solid surface. A target may be recombinant protein. In some exemplary embodiments, a target may be CD20, CD3, BCMA, PD-1, EGFR, CD28, CD38, TNF, PD-L1, or LAG3.

As used herein, the term “anti-drug antibodies” or “ADAs” refers to antibodies produced by the immune system of a subject that target epitopes on a therapeutic protein. A subset of ADAs are “neutralizing antibodies” or “NAbs”, which can bind to a therapeutic protein in a manner that inhibits or neutralizes its pharmacological activity. NAbs may affect the clinical efficacy of a therapeutic protein, and as such must be monitored when administering a therapeutic protein to a subject.

As used herein, the term “neutralizing agent” refers to a molecule that can interact with a therapeutic protein in a manner that inhibits or neutralizes its pharmacological activity. A neutralizing agent may be, for example, an oligonucleotide, such as an aptamer, or a protein, such as an antibody. Neutralizing agents may arise from a variety of sources, for example, by chemical synthesis, by recombinant production, or from the immune system of a subject. For simplicity, neutralizing antibodies (NAbs) produced by the immune system of a subject are the primary neutralizing agent discussed herein, but it should be understood that the methods of the invention may be applied to the detection of any neutralizing agent.

NAbs may be monitored using a variety of assays. NAb assays may be broadly divided into cell-based assays or non cell-based assays. The choice of cell-based assay versus non cell-based assay depends on the therapeutic protein, target, and application in question, and a person of skill in the art will be able to choose an assay according to their needs.

Cell-based assays comprise at least one type of cell. A therapeutic protein may bind to a target such that cellular events are impacted, which can then be measured as the output of therapeutic protein binding. Useful cellular events that result in a measurable signal or activity may include, for example, receptor phosphorylation, phosphorylation of downstream proteins in a signal transduction pathway, cytokine release, cell proliferation, cell death, production of a secondary protein, or any other cellular activity. Additionally or alternatively, a reporter gene that is expressed in response to cellular events caused by therapeutic protein binding to a target may be used; for example, a fluorescent protein such as luciferase, green fluorescent protein (GFP), or any variant thereof.

Measurement of signal generated by therapeutic protein binding to a target, and measurement of inhibition of that signal by NAbs, can be called a “direct” cell-based assay. Conversely, in an “indirect” cell-based assay, the binding of a therapeutic protein to a target inhibits a measurable signal, and the restoration of that signal is used to detect NAbs. For simplicity, discussion will be limited to direct cell-based assays, although the methods described herein may equally be applied towards indirect cell-based assays.

Disclosed herein are cell-based NAb assays comprising two types of cells which produce measurable cellular events when bridged by a therapeutic bispecific antibody. Each type of cell may present on its cell surface a target that is an antigen recognized by one arm of the bispecific antibody. The simultaneous binding of both targets bridges the two cells and produces downstream cellular events that can be measured as an indication of therapeutic protein binding. Examples of cells used for cell-based NAb assays include HEK293/hCD20 cells expressing human CD20, MOLP-8 cells endogenously expressing BCMA, and Jurkat/NFAT-Luc cells. Jurkat/NFAT-Luc cells express CD3 and the T-cell receptor (TCR) on their cell surface. When a bispecific antibody, for example a bispecific CD20×CD3 antibody or a bispecific BCMA×CD3 antibody, bridges this cell with a second cell, the TCR initiates a signal transduction pathway resulting in the expression of a luciferase reporter, generating a measurable signal. This signal may be reduced by the presence of NAbs or by competing drugs in the assay, as further described in the Examples.

It should be understood that many types of cells may be used in a cell-based assay of the invention according to the therapeutic protein and target being tested, provided that the cell expresses or can be modified to express a target, and/or can respond to the binding of a therapeutic protein and a target by producing a measurable signal or activity. Non-limiting examples of cells that can be used in the method of the invention include HEK293 cells, HEK293/hCD20 cells, HEK293/MfBCMA cells, HEK293/hBCMA cells, NCI-H929 cells, MOLP-8 cells, Jurkat cells, Jurkat/NFAT-Luc cells, Jurkat/NFAT-Luc/MfCD3 cells, and modified versions thereof.

Non cell-based assays can detect the presence of NAbs in the absence of cells. One type of non cell-based assay is called a competitive ligand binding (CLB) assay. CLB assays, or, as referred to herein, ligand binding assays, measure the binding of a therapeutic protein to a target, which may be, for example, a purified recombinant protein, or a native target associated with prepared cellular membrane. A target may be immobilized on a solid support, such as a microplate or beads, allowing for the capture of a labeled therapeutic protein, and detection of that label may be used to measure binding. NAbs in the sample will block the binding of the therapeutic protein to the target, reducing signal. Alternatively, a therapeutic protein may be immobilized to a solid surface while a soluble target is labeled, with the same principles applied otherwise. The label may be detectable and/or produce signal or activity by, for example, fluorescence, chemiluminescence, electrochemiluminescence, radioactivity, or affinity purification.

Measurement of signal generated by therapeutic protein binding to a target, and measurement of inhibition of that signal by NAbs, can be called a direct-binding assay. Conversely, in an indirect-binding assay, the binding of a therapeutic protein to a target inhibits a measurable signal, and the restoration of that signal is used to detect NAbs. For simplicity, discussion will be limited to direct-binding assays, although the methods described herein may equally be applied towards indirect-binding assays.

Disclosed herein are ligand binding NAb assays comprising biotinylated target, for example PD-1, immobilized onto an avidin-coated microplate, and co-incubated with ruthenylated therapeutic protein, for example cemiplimab. The binding of labeled cemiplimab to immobilized PD-1 allows for the detection of a signal which can be used to measure this binding. The presence of NAbs or competing drugs in the assay may reduce this signal, as further discussed in the Examples.

A second type of non cell-based assay is called an enzyme activity-based assay. Enzyme activity-based assays measure the ability of an enzyme drug product to catalyze a reaction biologically relevant to its mechanism of action, by converting a suitable substrate to a product. Enzyme activity may be measured by directly measuring the binding of the enzyme to its substrate, or by measuring the quantity of product produced. The presence of NAbs or competing drugs in the assay may be indicated by reduced binding or reduced production of the product. As such, the methods disclosed herein are also applicable to accurate quantitation of NAbs in an enzyme activity-based assay.

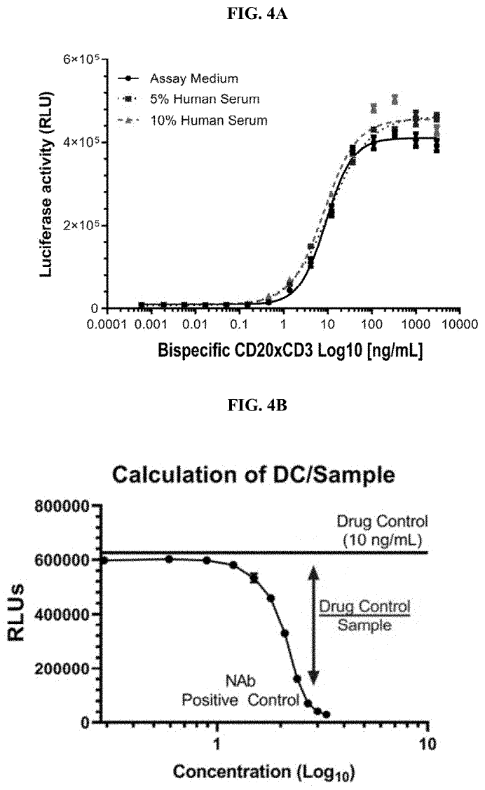

In order to detect the presence of NAbs in a sample, a NAb assay should include an experimental condition and a control condition. The experimental condition includes a sample that is being tested for the presence of NAbs. The control condition may be, for example, a negative control condition, which is known to not include NAbs. A signal or activity is generated in the NAb assay as a measure of therapeutic protein binding to a target, and a reduction of said signal in the experimental condition compared to the control condition is a measure of neutralization of the therapeutic protein, and thus the presence of NAbs in the experimental condition, as illustrated for example in FIG. 4B.

Conversely, a positive control condition could be known to include NAbs or another neutralizing agent, and could be used, for example, to validate a NAb assay or to calibrate its signal.

A change in signal between the experimental condition and the control condition may also be caused by interference from an interfering agent. Disclosed herein is a method of reducing said interference such that the presence of NAbs in a sample may be accurately detected.

As used herein, the term “interfering agent” refers to any molecule present in a NAb assay or sample matrix that may interfere with the accurate measurement of NAbs. Interference may be caused by association with NAbs, a therapeutic protein, a therapeutic protein target, or any component of a NAb assay. Examples of interfering agents may include a soluble target of the therapeutic protein, a protein with a similar sequence to the therapeutic protein that is thus targeted by the same NAb, or residual drug from a previous administration of the therapeutic protein.

A particular class of interfering agent may be a “competing drug” present in the sample matrix, which is not the therapeutic protein, but is capable of competitively binding to a component of a NAb assay, such as to a therapeutic protein target. A competing drug may be a residual drug previously administered to a subject. In some exemplary embodiments, a competing drug may competitively bind to therapeutic targets including, for example, CD20, CD3, BCMA, PD-1, EGFR, CD28, CD38, TNF, PD-L1, or LAG3. In some exemplary embodiments, a competing drug may be any of the drugs or drug candidates listed in Table 1. In some exemplary embodiments, a competing drug may be rituximab, pembrolizumab, nivolumab, ocrelizumab, obinutuzumab, ofatumumab, ibritumomab tiuxetan, tositumomab, ublituximab, cetuximab, daratumumab, or adalimumab.

As used herein, the term “mitigating agent” refers to any molecule that may bind to an interfering agent in order to reduce or prevent interference in a NAb assay and allow for accurate detection of NAbs in a sample. Any molecule that can specifically interact with an interfering agent and prevent its interference with NAbs, therapeutic proteins, targets, or other components of a NAb assay may be a suitable mitigating agent. A mitigating agent may be, for example, an oligonucleotide, such as an aptamer, or a protein, such as an antibody. In some exemplary embodiments, a mitigating agent may be a blocking antibody against a competing drug, such as an anti-rituximab blocking antibody, an anti-pembrolizumab blocking antibody, or an anti-nivolumab blocking antibody.

Also disclosed herein are kits for carrying out the method of the present invention. The kits of the invention allow a user to accurately detect the presence of NAbs in a sample by mitigating interference from competing drugs. The kits of the present invention may include, for example, a therapeutic protein, a target of said therapeutic protein, a mitigating agent, a means of producing a signal or activity as a measure of binding between said therapeutic protein and said target, and instructions for use of the kit. They may also include neutralizing agents that may be used as a positive control. They may additionally include competing drugs that may be used as a positive control.

Kits may be directed to cell-based or non cell-based NAb assays, or both. Kits directed towards cell-based NAb assays may comprise cells suitable for the expression of a target and for producing a signal or activity as a measure of therapeutic protein binding to said target, for example, HEK293/hCD20 cells, Jurkat/NFAT-Luc cells, MOLP-8 cells, or any other cell capable of expressing a target and/or capable of responding to the binding of a therapeutic protein to a target by producing a measurable signal or activity. A suitable target in a kit directed towards a cell-based NAb assay may be, for example, CD20, CD3, BCMA, EGFR, CD28, CD38, or a combination thereof. A suitable therapeutic protein may be, for example, a bispecific CD20×CD3 antibody, a bispecific BCMA×CD3 antibody, a bispecific EGFR×CD28 antibody, or a bispecific CD38×CD28 antibody.

Kits directed towards non cell-based NAb assays may comprise a solid support, for example a microplate or bead, capable of binding to a target and/or therapeutic protein, for example by being coated with avidin. They may additionally comprise a target and/or therapeutic protein capable of binding to said solid support, for example by being conjugated to biotin. They may further comprise a labeled target and/or therapeutic protein, for example a target and/or therapeutic protein labeled with ruthenium. A suitable target in a kit directed towards a non cell-based NAb assay may be, for example, PD-1, TNF, PD-L1, EGFR, CD20, CD38, or LAG3. A suitable therapeutic protein may be, for example, cemiplimab, or a monoclonal antibody directed against any of the aforementioned targets.

It is understood that the present invention is not limited to any of the aforesaid therapeutic protein(s), target(s), neutralizing agent(s), cell-based assay(s), cell type(s), non cell-based assay(s), reporter(s), label(s), interfering agent(s), competing drug(s), or mitigating agent(s), and any therapeutic protein(s), target(s), neutralizing agent(s), cell-based assay(s), cell type(s), non cell-based assay(s), reporter(s), label(s), interfering agent(s), competing drug(s), or mitigating agent(s) can be selected by any suitable means.

The present invention will be more fully understood by reference to the following Examples. They should not, however, be construed as limiting the scope of the invention.

### EXAMPLES

Materials and Methods. The present invention, when practiced by the person skilled in the art, may make use of conventional techniques in the field of pharmaceutical chemistry, immunology, molecular biology, cell biology, recombinant DNA technology, and assay techniques, as described in, for example, Sambrook et al. “Molecular Cloning: A Laboratory Manual”, 3^{rd} ed. 2001; Ausubel et al. “Short Protocols in Molecular Biology”, 5^{th} ed. 1995; “Methods in Enzymology”, Academic Press, Inc.; MacPherson, Hames and Taylor (eds.). “PCR 2: A practical approach”, 1995; “Harlow and Lane (eds.) “Antibodies, a Laboratory Manual” 1988; Freshney (ed.) “Culture of Animal Cells”, 4^{th} ed. 2000; “Methods in Molecular Biology” vol. 149 (“The ELISA Guidebook” by John Crowther) Humana Press 2001, and later editions of these treatises (e.g., “Molecular Cloning” by Michael Green (4^{th} Ed. 2012) and “Culture of Animal Cells” by Freshney (7^{th} Ed., 2015), as well as current electronic versions.

Reagents for carrying out the methods of the present invention, and aspects of the kits of the invention, include biotinylated PD-1 (targets); anti-rituximab antibodies α-Ritux Ab1, α-Ritux Ab2, and α-Ritux Ab3, anti-pembrolizumab antibodies, and anti-nivolumab antibodies (mitigating agents); anti-CD3 antibodies, anti-CD20 antibodies, anti-BCMA antibodies, anti-PD-1 antibodies, rituximab, pembrolizumab and nivolumab (competing drugs); and the bispecific antibody CD20×CD3, the bispecific antibody BCMA×CD3, and cemiplimab (therapeutic proteins); see, for example, U.S. Pat. Nos. 9,657,102 and 10,550,193, the entire teachings of which are herein incorporated by reference. Negative control antibodies, for example, hIgG1, hIgG4, are available from several commercial sources.

Cells suitable for carrying out the methods of the present invention, and aspects of the kits of the invention, include HEK293/hCD20, MOLP-8, Jurkat/NFAT-Luc and Jurkat/NFAT-Luc/MfCD3 cells, all of which are available from several commercial sources.

Luciferase assays are carried out according to guidelines from the manufacturer; see for example, Promega and ThermoFisher.

### Example 1. Cell-Based Assay Design for Detecting Neutralizing Antibodies (NAbs) Against a Therapeutic Protein

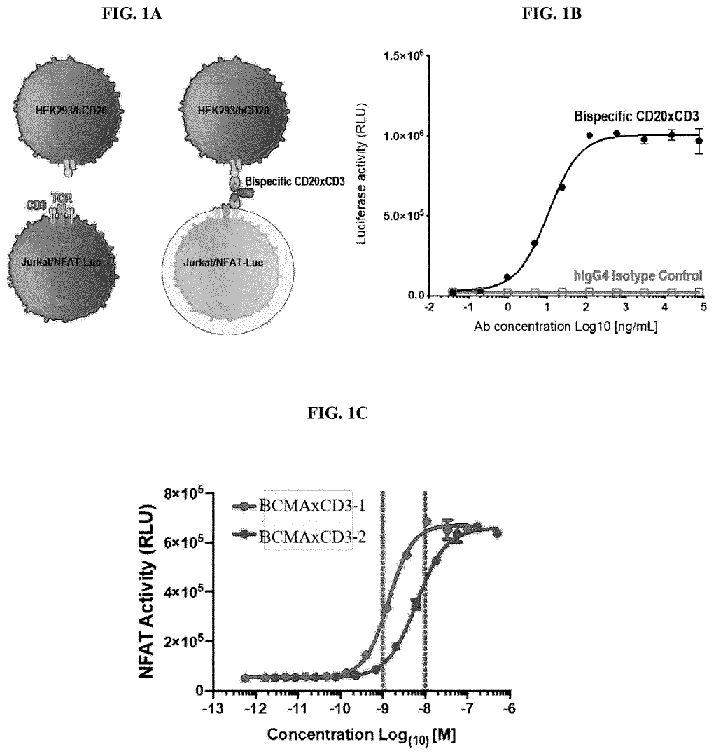

This example shows the experimental design of cell-based neutralizing antibody (NAb) assays of the invention for evaluating therapeutic protein candidates. Briefly, human immortalized B cells engineered to express the cell surface human antigen CD20 were prepared (designated HEK293/hCD20). These cells represent the “target cells” of the assay that mimic human cancer cells expressing CD20. In addition, human immortalized T-cells expressing the T-cell receptor (TCR) and cell surface antigen CD3 were prepared and engineered to express a reporter gene (luciferase) under the control of a TCR/CD3 inducible promoter (Nuclear factor of activated T-cells (NFAT)). These Jurkat/NFAT-Luc cells represent the “reporter cells” of the assay that mimic a patient's immune cells capable of engaging and potentially eliminating a CD20 expressing cancer cell via a cell-mediated cytotoxicity response when bridged with a drug antibody, such as a bispecific CD20×CD3 antibody, as shown in FIG. 1A.

The addition of antibodies that bind CD20 and CD3 (the bispecific CD20×CD3 drug antibody), mediating the clustering of the T-cell receptor (TCR) on the reporter cell, results in the expression of the luciferase reporter gene and provides for a robust dose-dependent luciferase signal, as shown in FIG. 1B. The addition of a hIgG4 isotype control antibody did not produce luciferase activity, as indicated by the open squares in FIG. 1B.

Another cell-based NAb assay was designed using Jurkat/NFAT-Luc cells as reporter cells as described above, in combination with MOLP-8 cells as target cells. MOLP-8 is a multiple myeloma cell line that endogenously expresses the cell surface protein B cell maturation antigen (BCMA). Bispecific BCMA×CD3 antibodies can bridge the reporter and target cells, mediating the clustering of the TCR on the reporter cell, leading to expression of the luciferase reporter gene and dose-dependent luciferase signal, as shown in FIG. 1C. Two BCMA×CD3 antibodies were tested, with the dotted lines indicating the concentration used in subsequent assays.

These results show that the cell-based assays of the invention provide a robust dose response curve and predictably respond to positive and negative controls.

### Example 2. Detection of NAbs Against a Therapeutic Protein Using a Cell-Based NAb Assay

This example shows further proof of concept of the experimental design of the NAb assay of the present invention. In a cell-based NAb assay, NAbs against a therapeutic protein inhibit binding of the therapeutic protein to its target and/or reporter cells, and thereby eliminate reporter signal. The reduction of reporter signal or activity in the NAb assay is a measure of the presence of NAbs in the sample.

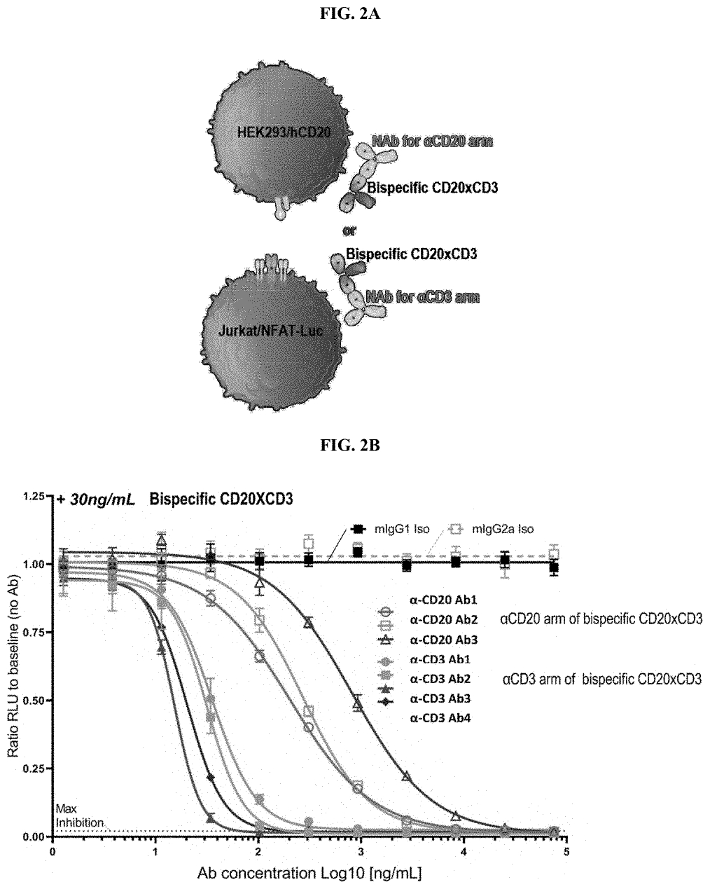

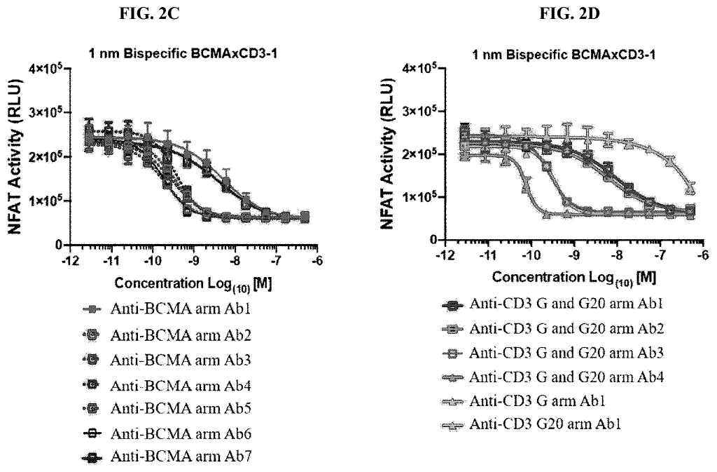

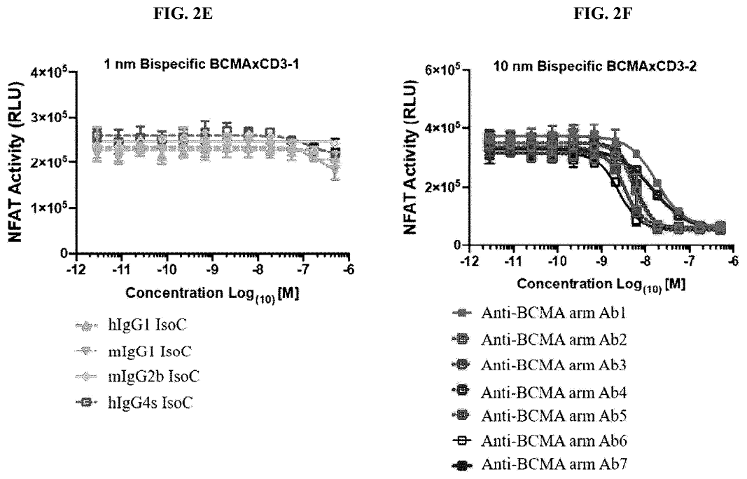

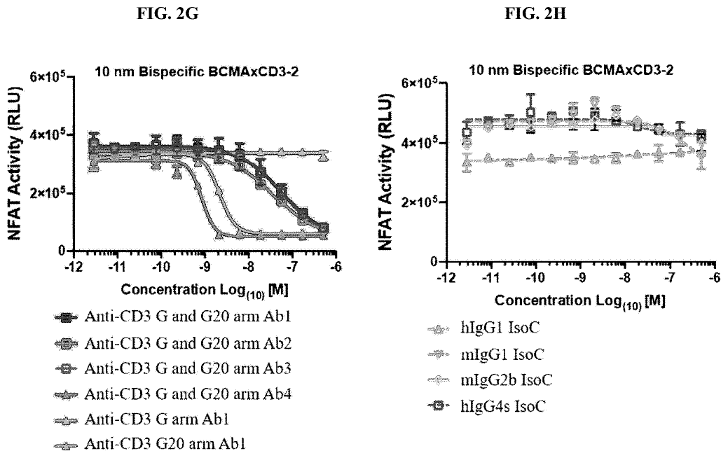

For example, FIG. 2A illustrates the action of NAbs against a bispecific CD20×CD3 drug antibody, wherein binding of NAbs against the anti-CD20 arm or anti-CD3 arm of the bispecific antibody interrupts binding to CD20 or CD3 respectively, eliminating luciferase activity. To further validate this cell-based NAb assay, surrogate NAbs were added to the NAb assay, targeting either the anti-CD20 arm or anti-CD3 arm of the bispecific CD20×CD3 drug antibody. Addition of NAbs caused a decrease in luciferase activity in a dose-dependent manner, as shown in FIG. 2B.

The effectiveness of this cell-based NAb assay was further validated for use with two bispecific BCMA×CD3 drug antibodies. Surrogate NAbs were added to the NAb assay, targeting either the anti-BCMA arm or anti-CD3 arm of the two bispecific BCMA×CD3 drug antibodies. Addition of NAbs caused a decrease in luciferase activity in a dose-dependent manner, as shown in FIGS. 2C, 2D, 2F and 2G. Addition of isotype controls had no effect on luciferase activity, as shown in FIGS. 2E and 2H.

These results show that the assay of the present invention reliably measures the presence of neutralizing antibodies against a therapeutic protein in a dose-dependent manner.

### Example 3. Cell-Based NAb Assay Interference by a Competing Drug

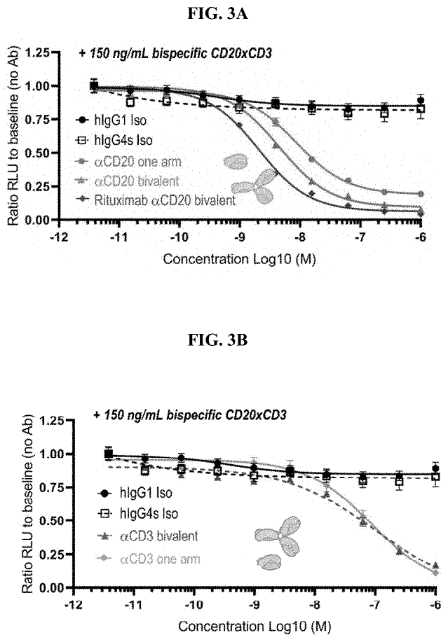

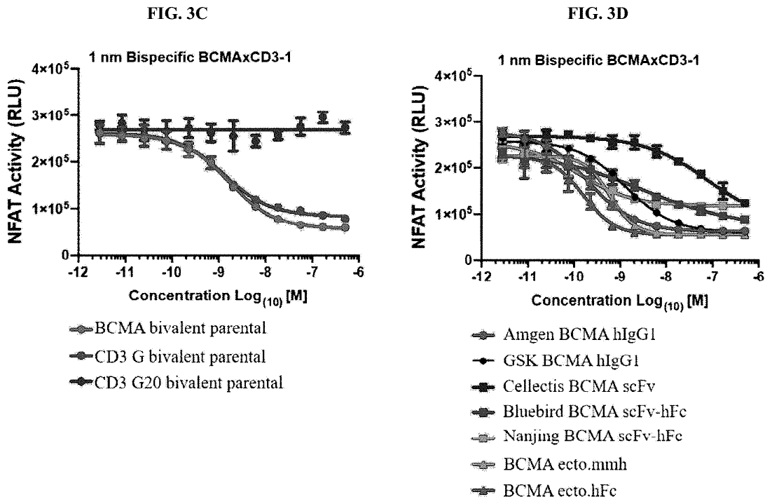

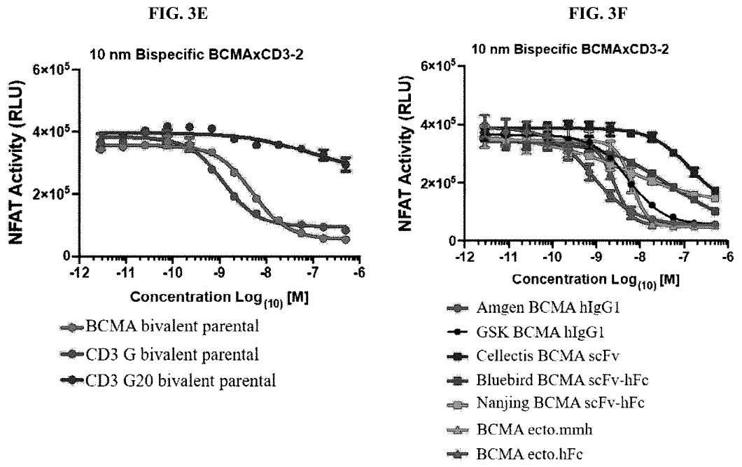

NAb assays may be susceptible to false positive or false negative results due to interference from matrix components. One potential source of interference is a second drug that competitively binds to the target of the therapeutic protein being tested. As a proof of concept of this type of interference, NAb assays for a bispecific CD20×CD3 drug antibody were conducted with the addition of competing antibodies against either CD20 or CD3, as shown in FIG. 3A and FIG. 3B. The addition of a competing drug caused a dose-dependent reduction in luciferase activity, mimicking the reduction in luciferase activity caused by surrogate NAbs and therefore producing a false positive result.

Interference from competing drugs was also seen in NAb assays for two bispecific BCMA×CD3 drug antibodies. The addition of bivalent parental antibodies against BCMA or CD3 caused a decrease in luciferase signal in both assays, as shown in FIGS. 3C and 3E. The addition of various clinical candidate antibodies against BCMA also caused a decrease in luciferase signal in both assays, as shown in FIGS. 3D and 3F.

These results demonstrate proof of concept that the presence of a competing second drug can produce a false positive result in a cell-based NAb assay.

### Example 4. Addition of Human Serum to a Cell-Based NAb Assay

As discussed above, NAb assays may be susceptible to interference from matrix components. To test the resilience of the NAb assay of the invention to potential interference, the NAb assay for a bispecific CD20×CD3 drug antibody was conducted with the addition of drug-naïve human serum as shown in FIG. 4A. Luciferase activity was unaffected by the addition of human serum, demonstrating the resilience of the NAb assay of the invention to interference from human serum components and therefore suitability for clinical application.

FIG. 4B demonstrates a simple representation of “NAb assay signal”. The relative presence of NAbs in a sample is quantitated by dividing luciferase activity induced with a drug control over luciferase activity induced in an experimental sample. Luciferase activity is reduced in a dose-dependent manner in the presence of NAbs, leading to a higher NAb assay signal.

### Example 5. Cell-Based NAb Assay Interference in Clinical Samples

The NAb assay of the present invention was used to test 60 drug-naïve human samples from a clinical trial for the presence of NAbs against a bispecific CD20×CD3 drug antibody, as shown in FIG. 5. Although the tested patients had not been exposed to the drug antibody, many samples showed a false positive result for NAbs.

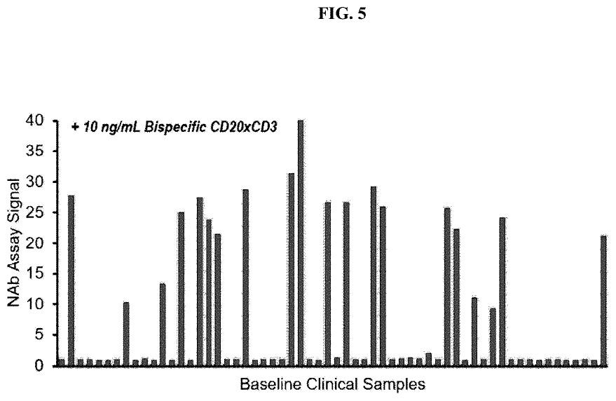

As discussed in Example 3, one possible source of a false positive signal in a NAb assay is a competing second drug. Many patients in this clinical trial had a history of prior anti-CD20 therapy. In order to assess whether a competing anti-CD20 drug may be responsible for the false positive results of the NAb assay, a subset of 17 human samples were tested for the presence of rituximab, an anti-CD20 antibody, using a commercially available ELISA. The presence of rituximab correlated with false positive NAb assay signal, as shown in FIG. 6.

These results demonstrate that interference from a residual competing drug may result in false positive results in a NAb assay in a clinical application, and must be addressed in order to accurately detect NAbs against a therapeutic protein.

### Example 6. Mitigation of Cell-Based NAb Assay Interference by a Competing Drug

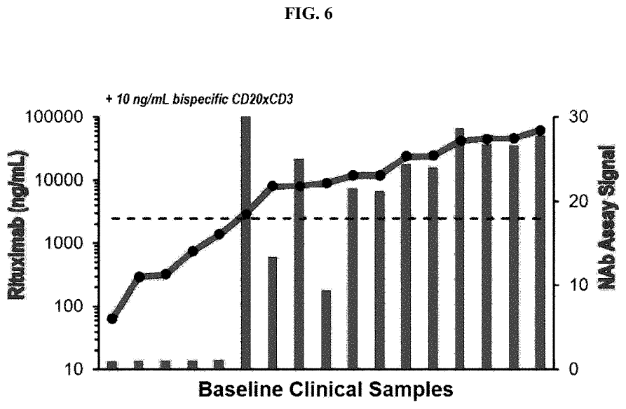

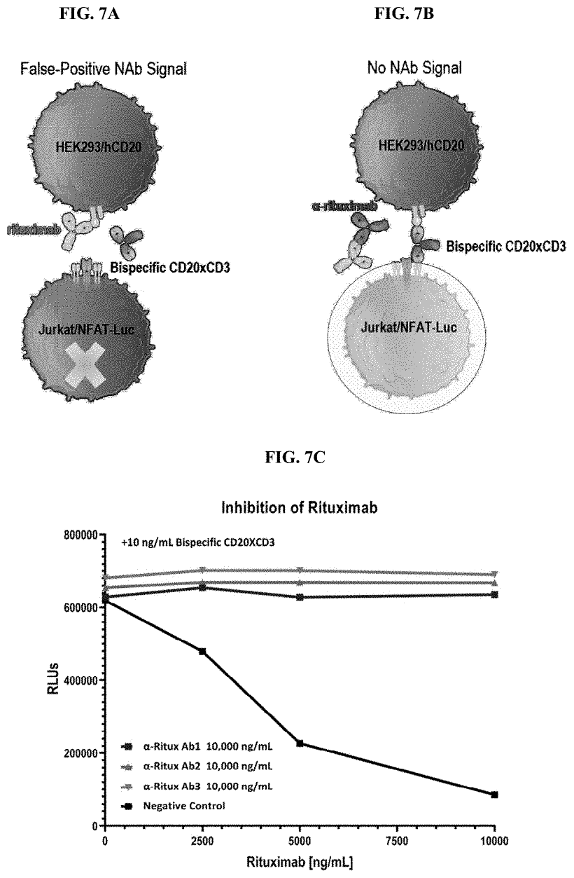

As described above, the presence of a competing drug may interfere with the binding of a therapeutic protein to its target in a NAb assay, resulting in reduction of reporter activity and a false positive NAb assay signal. This is illustrated in FIG. 7A, using the example of a bispecific CD20×CD3 drug antibody as the therapeutic protein and rituximab, an anti-CD20 antibody, as the competing drug. In order to accurately detect NAbs against a therapeutic protein in the presence of a competing drug, binding of the competing drug to the mutual target must be mitigated. This is illustrated in FIG. 7B, using the example of an anti-rituximab antibody as a mitigating agent preventing interference from the competing drug and allowing the accurate detection of NAbs against the therapeutic protein.

Blocking antibodies against rituximab were tested for their ability to mitigate interference in the NAb assay of the present invention. Anti-rituximab antibodies were co-incubated in serum spiked with rituximab and added to a NAb assay, as shown in FIG. 7C. Addition of anti-rituximab antibodies restored luciferase activity, eliminating the false positive NAb assay signal caused by rituximab.

These results demonstrate that the use of a mitigating agent against a competing drug can eliminate false positive NAb assay signal and allow for accurate detection of NAbs against a therapeutic protein.

### Example 7. Mitigation of Cell-Based NAb Assay Interference by a Competing Drug in Clinical Samples

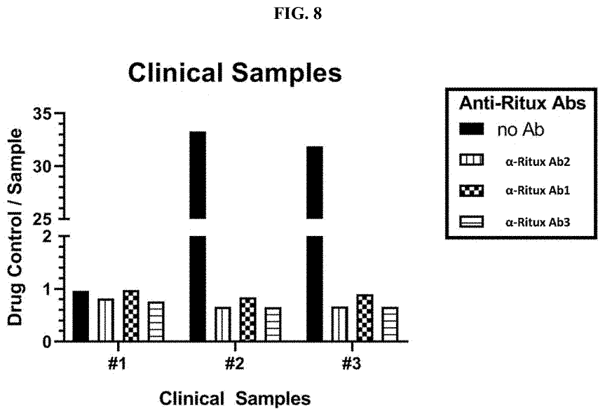

As shown in Example 5, many drug-naïve human samples from a clinical trial yielded false-positive NAb assay signal when tested for NAbs against a bispecific CD20×CD3 drug antibody, potentially due to the presence of a competing drug, the anti-CD20 antibody rituximab. In order to mitigate interference from rituximab, NAb assays were conducted using clinical samples with the addition of anti-rituximab blocking antibodies, as shown in FIG. 8. Sample #1 is a control sample with low NAb assay signal. Samples #2 and #3 showed high false positive NAb assay signal. The addition of anti-rituximab antibodies eliminated the false positive NAb assay signal.

These results confirm that a residual competing drug in clinical samples, in this case rituximab, can interfere with a NAb assay and render the results of the NAb assay inaccurate. They further demonstrate that mitigating agents against a competing drug can eliminate false positive NAb assay signal in a clinical application. The use of mitigating agents against a competing drug allows for the accurate detection of NAbs against the therapeutic protein being tested.

### Example 8. Ligand Binding Assay Design for Detecting NAbs Against a Therapeutic Protein

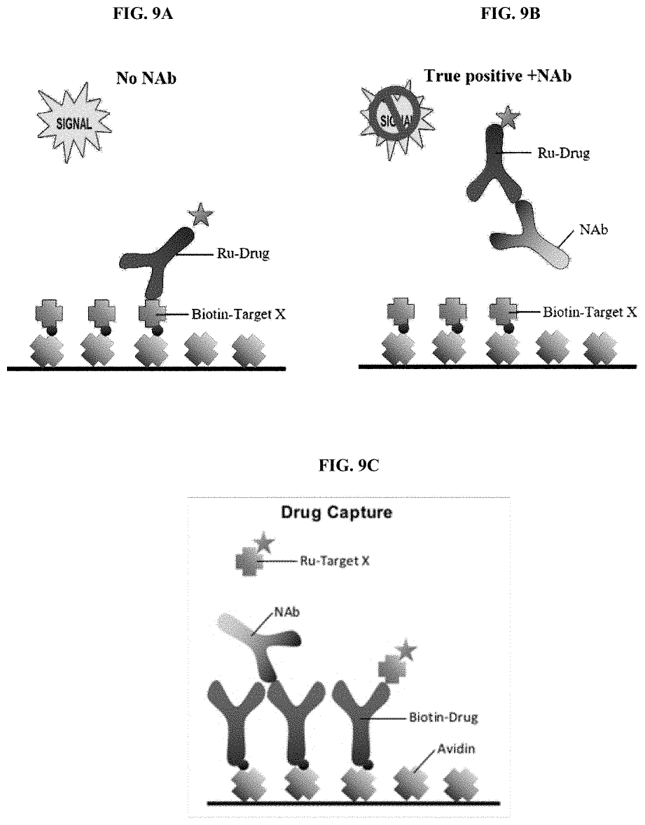

This example shows the experimental design of a ligand binding NAb assay of the invention for evaluating a therapeutic protein candidate. An exemplary embodiment of the invention comprises a target-capture ligand binding NAb assay. Briefly, samples are incubated with a biotinylated target and transferred to an avidin-coated microplate. Ruthenylated drug is added to the microplate in a subsequent step. In the absence of NAbs, ruthenium-labeled drug binds to the immobilized biotin-target, generating signal in the assay, as shown in FIG. 9A. In the presence of NAbs, ruthenium-labeled drug cannot bind to the biotin-target, resulting in inhibition of the assay signal, as shown in FIG. 9B.

Additional ligand binding NAb assays may be suitable for assessing NAbs against a therapeutic protein. For example, instead of a target-capture design, a ligand binding assay may be designed for drug-capture: the therapeutic protein of interest is immobilized, and the target is labeled for the generation of assay signal. FIG. 9C illustrates a ligand binding assay design with biotinylated drug immobilized on an avidin-coated microplate, ruthenylated target generating assay signal, and NAbs against the immobilized drug blocking binding to the target and thereby inhibiting the assay signal.

### Example 9. Ligand Binding NAb Assay Interference by a Competing Drug

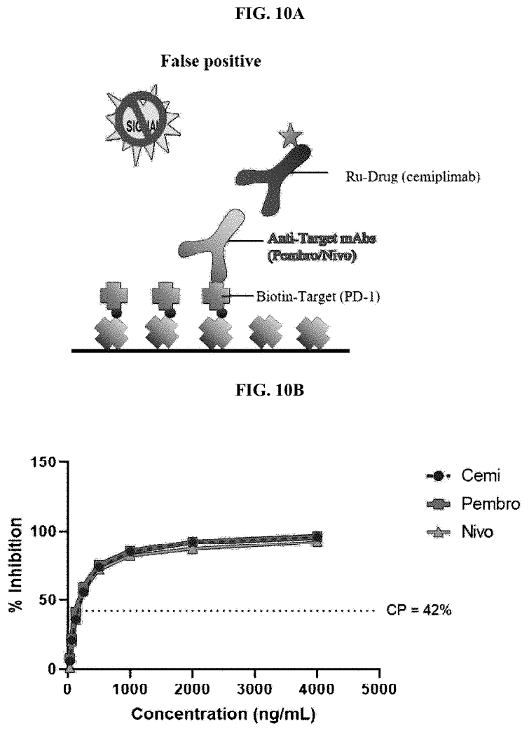

Like the cell-based NAb assay described above, a ligand binding NAb assay may be susceptible to false positive or false negative results due to interference from matrix components. One potential source of interference is a second drug that competitively binds to the target of the therapeutic protein being tested, as shown in FIG. 10A. For example, the drug antibodies cemiplimab, pembrolizumab and nivolumab share the same drug target, PD-1. If a clinical sample is tested for NAbs against cemiplimab, using the binding of ruthenylated cemiplimab to biotinylated PD-1 to generate signal, any residual pembrolizumab, nivolumab, or unlabeled cemiplimab in the clinical sample would competitively bind to the target, inhibiting the assay signal and causing a false positive result for the presence of NAbs.

As a proof of concept, increasing concentrations of cemiplimab, pembrolizumab or nivolumab were added to a target-capture NAb assay for NAbs against cemiplimab, as shown in FIG. 10B. Concentrations of cemiplimab, pembrolizumab or nivolumab above 125 ng/mL inhibited signal from ruthenylated cemiplimab, producing a false positive NAb assay signal.

These results demonstrate that the presence of a competing drug can result in a false positive ligand binding NAb assay signal, and must be addressed in order to accurately detect NAbs against a therapeutic protein.

### Example 10. Mitigation of Ligand Binding NAb Assay Interference by a Competing Drug

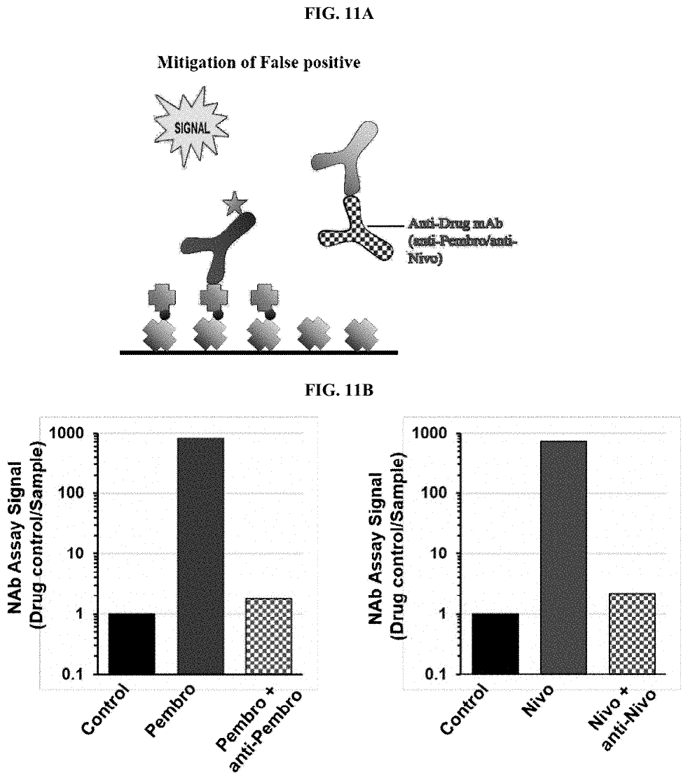

As described above, the presence of a competing drug may interfere with the binding of a therapeutic protein to its target in a ligand binding NAb assay, resulting in reduction of signal and a false positive NAb assay signal. In order to accurately detect NAbs against a therapeutic protein in the presence of a competing drug, binding of the competing drug to the mutual target must be mitigated. This is illustrated in FIG. 11A, using the example of an anti-pembrolizumab or anti-nivolumab antibody as a mitigating agent preventing interference from the competing drug and allowing the accurate detection of NAbs against the therapeutic protein.

Blocking antibodies against pembrolizumab and nivolumab were tested for their ability to mitigate interference in the NAb assay of the invention. Anti-pembrolizumab or anti-nivolumab antibodies were co-incubated in samples spiked with pembrolizumab or nivolumab, respectively, and added to a ligand binding NAb assay, as shown in FIG. 11B. Addition of mitigating agents against the competing drugs eliminated the false positive NAb assay signal caused by competitive binding to the target.

These results demonstrate that the use of a mitigating agent against a competing drug can eliminate false positive NAb assay signal in a ligand binding assay, and allow for accurate detection of NAbs against a therapeutic protein.

What is claimed is:

1. A method for detecting a neutralizing agent to a therapeutic protein in a sample, comprising:
(a) contacting said sample having said neutralizing agent and a competing drug to said therapeutic protein, a target of said therapeutic protein, and a mitigating agent;
(b) measuring a binding of said therapeutic protein to said target; and
(c) comparing the result of (b) to a control measurement to detect said neutralizing agent.

2. The method of claim 1, wherein said control measurement includes measuring binding of said therapeutic protein to said target in the absence of a neutralizing agent.

3. The method of claim 1, wherein said neutralizing agent is a neutralizing antibody.

4. The method of claim 1, wherein said therapeutic protein is selected from a group consisting of an antibody, a soluble receptor, an antibody-drug conjugate, and an enzyme.

5. The method of claim 1, wherein said therapeutic protein is a monoclonal antibody.

6. The method of claim 5, wherein said monoclonal antibody is selected from a group consisting of an anti-PD-1 antibody, an anti-TNF antibody, an anti-PD-L1 antibody, an anti-EGFR antibody, an anti-CD20 antibody, an anti-CD38 antibody, and an anti-LAG3 antibody.

7. The method of claim 1, wherein said therapeutic protein is a bispecific antibody.

8. The method of claim 7, wherein said bispecific antibody is selected from a group consisting of a CD20×CD3 antibody, a BCMA×CD3 antibody, a EGFR×CD28 antibody, and a CD38×CD28 antibody.

9. The method of claim 1, wherein said therapeutic protein is immobilized to a solid support.

10. The method of claim 1, wherein said therapeutic protein is labeled for detection.

11. The method of claim 10, wherein said label is detectable by fluorescence, chemiluminescence, electrochemiluminescence, radioactivity, or affinity purification.

12. The method of claim 11, where said label comprises ruthenium.

13. The method of claim 1, wherein said target is an antigen, a receptor, a ligand, or an enzymatic substrate.

14. The method of claim 1, wherein said target is a cell surface protein.

15. The method of claim 1, wherein said target is a recombinant protein.

16. The method of claim 1, wherein said target is expressed by a cell.

17. The method of claim 16, wherein said cell is a HEK293 cell, a MOLP-8 cell, a Jurkat cell, or a modified version thereof.

18. The method of claim 1, wherein said target is immobilized to a solid support.

19. The method of claim 1, wherein said target is labeled for detection.

20. The method of claim 19, wherein said label is detectable by fluorescence, chemiluminescence, electrochemiluminescence, radioactivity, or affinity purification.

21. The method of claim 1, wherein said target is an enzymatic substrate.

22. The method of claim 1, wherein said target is CD20, CD3, BCMA, PD-1, EGFR, CD28, CD38, TNF, PD-L1, or LAG3.

23. The method of claim 1, additionally comprising a second target.

24. The method of claim 1, wherein said competing drug is a monoclonal antibody.

25. The method of claim 24, wherein said competing drug is rituximab, pembrolizumab, nivolumab, ocrelizumab, obinutuzumab, ofatumumab, ibritumomab tiuxetan, tositumomab, ublituximab, cetuximab, daratumumab, or adalimumab.

26. The method of claim 1, wherein said competing drug is a bispecific antibody.

27. The method of claim 1, wherein said mitigating agent is a monoclonal antibody.

28. The method of claim 1, comprising using two, three, four or more mitigating agents.

29. The method of claim 1, wherein a binding of said therapeutic protein to said target is measured by measuring receptor phosphorylation, phosphorylation of downstream proteins in a signal transduction pathway, cytokine release, cell proliferation, cell death, or production of a secondary protein.

30. The method of claim 1, wherein a binding of said therapeutic protein to said target is measured by the expression of a reporter gene.

31. The method of claim 30, wherein said reporter gene is luciferase.

32. The method of claim 1, further comprising a pre-treatment step of contacting said sample to said mitigating agent prior to contacting said sample to said therapeutic protein or said target.

33. A kit, comprising:
(a) a therapeutic protein;
(b) a target of said therapeutic protein;
(c) a neutralizing agent against said therapeutic protein;
(d) a competing drug; and
(e) a mitigating agent.

34. The kit of claim 33, further comprising cells that express said target.

35. The kit of claim 34, further comprising cells that produce a measurable activity or signal in response to the binding of said therapeutic protein to said target.

36. The kit of claim 35, wherein said activity is the expression of luciferase.

37. The kit of claim 33, wherein said target is immobilized to a solid support.

38. The kit of claim 33, further comprising a label affixed to said therapeutic protein.

39. The kit of claim 38, wherein said label comprises ruthenium.
